SEQUENCE LISTING (1 sequence)

SEQ ID NO: 1 - DNA, 39 bp, Artificial Sequence
  Description of Artificial Sequence Primer
ggccagtgaattgtaatacgactcactatagggaggcgg

(19) United States
(12) Patent Application Publication
(10) Pub. No.: US 2011/0044946 A1
(43) Pub. Date: Feb. 24, 2011

(54) GENETEC REGULATION OF HOST DEFENSE MECHANISMS BY MUCOSAL EXPOSURE TO NATURAL INTERFERON ALPHA SPECIES

(76) Inventors: William A. Carter, Spring City, PA (US); David Strayer, Bryn Mawr, PA (US)

Correspondence Address: NIXON & VANDERHYE, PC, 901 NORTH GLEBE ROAD, 11TH FLOOR, ARLINGTON, VA, 22203, US

(21) Appl. No.: 12/735,753

(22) PCT Filed: Feb. 17, 2009
(86) PCT No.: PCT/US2009/000960

§ 371 Date: Aug. 13, 2010

Related U.S. Application Data
(60) Provisional application No. 61/051,366, filed on May 8, 2008.
(60) Provisional application No. 61/029,308, filed on Feb. 15, 2008.

(51) Int. Cl. A61K 38/21; A61P 31/04; A61P 31/12; A61P 35/00; A61P 37/02; A61P 25/28; A61P 33/02

(52) U.S. Cl. 424/85.7

(57) ABSTRACT

A mixture of α-interferons (IFN-α) is used in vitro or in vivo as an antimicrobial agent or anticancer agent. It may be administered by topical application to oral or nasal, and/or buccal mucosa to combat the effects of infection by bacteria or protozoa, cancer, or other pathogenic disease process (e.g., autoimmune or neurodegenerative disease).

### CROSS-REFERENCE TO RELATED APPLICATION

This application claims the benefit of Application No. 61/029,308, filed Feb. 15, 2008, and Application No. 61/051,366, filed May 8, 2008.

### BACKGROUND OF THE INVENTION

This invention relates to use of α-interferon (IFN-α) composition, which contains a mixture of naturally occurring α-interferons and other optional components, to activate an immune response combating cancer or infection by bacteria or protozoa. Methods of medical treatment and processes for manufacturing medicaments are provided.

The immune system consists of innate immunity, which does not require antigen selection and T-cell help, and adaptive immunity, which requires a longer period of time for induction after contact with antigen. Thus, innate immunity can react quickly but nonspecifically against (i) foreign invaders such as bacteria and protozoa or (ii) cell transformation in precancerous and cancerous conditions.

Type 1 interferons (notably those of IFN-α) act to synchronize the timing and relative amounts of various soluble immune mediators (e.g., cytokines) during an immune response. The absence of IFN-α can lead to dysregulation of innate immunity (also called “cytokine storm”), resulting in overwhelming pneumonia. Post mortem examination showed that these inflammatory cytokines (e.g., tumor necrosis factor-alpha or TNF-α) can injure the lungs, and possibly lead to death, through such immune dysregulation. Immune dysregulation can be normalized by very small doses of interferon-α applied to the oral, nasal, or buccal mucosa, either before or during early stages of otherwise overwhelming viral infections. We have now discovered the utility of low doses of IFN-α applied to the oral, nasal, or buccal mucosa to treat cancer or infection by bacteria or protozoa. Surprisingly, unlike other chemotherapeutic agents that are only effective against a specific microbe or cancer (e.g., antibacterial penicillin, antimalarial chloroquine, and anticancer methotrexate), a low dose of oral IFN-α has broad application as an antimicrobial chemotherapeutic agent effective in treatment of bacteria, protozoa, and cancer by acting directly on the immune system.

U.S. Pat. No. 4,497,795 discloses increasing the efficiency of food utilization by administering human alpha interferon orally in a dosage between about 0.10 and 1.5 International Units (IU)/pound of body weight per day.

U.S. Pat. No. 5,830,456 discloses contacting the oral and/or pharyngeal mucosa with interferon dosed at less than 5 IU/pound of body weight per day “to potentiate disease-corrective immune responses in vertebrates afflicted with immuno-resistant disease states characterized by apparent hyperactive or hypoactive immune system function.” Examples of treatable diseases listed therein are “neoplastic disease, hyperallergenicity, immunoresistant or immunodebilitating viral infections, and autoimmune disorders characterized by chronic tissue degenerative inflammation.” Human patients with acute rheumatoid arthritis, multiple sclerosis, asthma, acne, malignant lymphoma, mesothelioma, or apthous stomatitis were treated with interferon alpha. Interferon alpha was administered at 0.7 IU/pound of body weight twice daily in a buffered solution having a concentration such that a single dosage could be administered in a volume of about 1 ml to about 20 ml of liquid. The patient held the liquid dose in the mouth for a period of time up to about one minute.

U.S. Pat. No. 6,361,769 discloses using interferon at 1500 IU/day to 20×10⁶ IU/day applied to oral or oropharyngeal mucosa to treat autoimmune, mycobacterial, neurodegenerative, parasitic, or viral conditions.

But the prior art does not teach or suggest a highly purified mixture of different human interferons as used in our invention, which achieves systemic activation and modulation of a broad spectrum of genes related to immune regulation.

Therefore, it is an objective of the invention to provide treatment for a patient in need of an antibacterial agent, antiprotozoal agent, and/or antiproliferative agent. Methods for treating subjects and processes for making medicaments, especially involving infectious disease and/or cell transformation, are provided. Other objectives and advantages are described below.

### SUMMARY OF THE INVENTION

It is an objective to treat a subject (e.g., human or animal) with an incipient or established bacterial or protozoal infection, and thereby bring the immune response to the infection back to balance (i.e., remodulation) and to avoid the devastation of a dysregulated immune response. In particular, the level of tumor necrosis factor-alpha (TNF-α) may be reduced and lung pathology may be avoided (e.g., measured as a decrease in the number or severity of pneumonia-like indications such as oxidative damage, pulmonary edema, and hemorrhage).

A subject may be infected with a bacterium or protozoan. A pharmaceutical composition, which is comprised of a low dose of an IFN-α cocktail, is administered to the subject. Infection of the subject is reduced or eliminated thereby as assayed by decreased recovery time, increased immunity (e.g., increased antibody titer, lymphocyte proliferation, or killing of infected cells), decreased division or growth of the infectious agent, or any combination thereof as compared to the subject not treated with a low dose of the IFN-α cocktail.

It is another objective to treat a subject (e.g., human or animal) affected by a precancerous or cancerous condition (e.g., neoplasm or tumor), and thereby up regulate host defense responses to transformed or immortalized cells and to control abnormal cell proliferation.

A subject may be afflicted by abnormal cell proliferation (e.g., neoplasm or tumor, other transformed cells). A pharmaceutical composition, which is comprised of a low dose of an IFN-α cocktail, is administered to the subject. Disease in the subject is reduced or eliminated thereby as assayed by improved morbidity or mortality, increased immunity (e.g., increased antibody titer, lymphocyte proliferation, or killing transformed cells), decreased division or growth of proliferating or transformed cells, or any combination thereof as compared to the condition of a subject not treated with a low dose of the IFN-α cocktail.

Another objective is to treat a subject (e.g., human or animal) infected by a bacterium or protozoa or affected by a pathogenic disease process (i.e., auto-immune or neurodegenerative disease) susceptible to treatment by the specific genes regulated by type 1 interferons. A pharmaceutical composition, which is comprised of a low dose of an IFN-α cocktail, is administered to the subject. Disease in the subject is reduced or eliminated thereby by increasing expression of underexpressed genes and decreasing expression of overexpressed genes as compared to the condition of a subject not treated with a low dose of the IFN-α cocktail.

An IFN-α cocktail may be a mixture of different human interferon-alpha proteins. It includes human interferon-alpha proteins of at least three, at least six, at least 10, at least 14, or at least 18 different subtypes (i.e., type 1 species), which are produced naturally or recombinantly in mammalian cells. The IFN-α proteins may be purified from human blood product, medium in which human virally-infected leukocytes were cultured, or medium or a body fluid (e.g., blood, milk, urine) in which cultured or transgenic mammalian cells recombinantly express human IFN-α genes. The purified cocktail thus contains naturally-derived human sequences of different IFN-α subtypes modified in accordance with mammalian glycosylation patterns. The purified cocktail is preferably administered by topical application to oral, nasal, and/or buccal mucosa (i.e., oropharyngeal application) instead of enteral or parenteral administration, or intranasal or intratracheal inhalation. Administration of the purified cocktail at least remodulates (i.e., increasing or decreasing back to the normal levels of a healthy subject) cytokine production or co-stimulatory molecule signaling which had been initiated by the microbe or processes resulting in autoimmunity or neurodegeneration in the subject.

Also provided are processes for using and making medicaments. It should be noted, however, that a claim directed to the product is not necessarily limited to these processes unless the particular steps of the process are recited in the product claim.

Further aspects of the invention will be apparent to a person skilled in the art from the following description of specific embodiments and the claims, and generalizations thereto.

### DESCRIPTION OF SPECIFIC EMBODIMENTS OF THE INVENTION

An infection by a bacterium or protozoan may be treated. It may infect a human or animal subject. The infection may be incipient or established. The microbe may be a bacterium or protozoan, especially those that can cause disease (i.e., pathogenic microbes). Here, the terms “microbe” and “microorganism” are used interchangeably.

The bacterium may be a species of the genus Bacillus (e.g., B. anthracis, B. cereus), Bartonella (B. henselae), Bordetella (e.g., B. pertussis), Borrelia (e.g., B. burgdorferi), Brucella (e.g., B. abortus), Campylobacter (e.g., C. jejuni), Chlamydia (e.g., C. pneumoniae), Clostridium (e.g., C. botulinum, C. difficile, C. perfringens, C. tetani), Corynbacterium (e.g., C. amycolatum, C. diphtheriae), Escherichia (e.g., E. coli O175:H7), Haemophilus (e.g., H. influenzae), Heliobacter (e.g., H. pylori), Klebsiella (K. pneumoniae), Legionella (e.g., L. pneumophila), Listeria (e.g., L. monocytogenes), Mycobacterium (e.g., M. avium, M. bovis, M. branderi, M. leprae, M. tuberculosis), Mycoplasma (e.g., M. genitalium, M. pneumoniae), Neisseria (e.g., N. gonorrheae, N. meningitidis), Pneumocystis (e.g., P. carinii), Pseudomonas (P. aeruginosa), Rickettsia, (e.g., R. rickettsia, R. typhi), Salmonella (e.g., S. enterica), Shigella (e.g., S. dysenteriae), Staphylococcus (e.g., S. aureus, S. epidermidis), Streptococcus (e.g., S. pneumoniae, S. pyogenes), Treponema (e.g., T. pallidum), Vibrio (e.g., V. cholerae, V. vulnificus), or Yersinia (e.g., Y. pestis). These include Gram-negative or Gram-positive bacteria, chlamydia, spirochetes, mycobacteria, and mycoplasmas.

The protozoan may be a species of the genus Cryptosporidium (e.g., C. hominis, C. parvum), Entamoeba (e.g., E. histolytica), Giardia (e.g., G. intestinalis, G. lamblia), Leishmania (e.g., L. amazonensis, L. braziliensi, L. donovani, L. mexicana, L. tropica), Plasmodium (e.g., P. falciparum, P. vivax), Toxoplasma (e.g., T. gondii), or Trypanosoma (e.g., T. bruci, T. cruzi).

Cells of a subject undergoing abnormal proliferation may be a neoplasm or tumor (e.g., carcinoma, sarcoma, leukemia, lymphoma), especially cells transformed by a tumor virus (e.g., DNA or RNA virus carrying a transforming gene or oncogene) or otherwise infected by a virus associated with cancer. For example, Epstein-Barr virus (EBV) is associated with nasopharyngeal cancer, Hodgkin's lymphoma, Burkitt's lymphoma, and other B-cell lymphomas; human hepatitis B and C viruses (HBV and HCV) are associated with liver cancer; human herpesvirus 8 (HHV8) is associated with Kaposi's sarcoma; human papillomaviruses (e.g., HPV6, HPV11, HPV16, HPV18, or combination thereof) are associated with cervical cancer, anal cancer, and genital warts; and human T-lymphotrophic virus (HTLV) is associated with T-cell leukemia or lymphoma. Cancers include those originating from the gastrointestinal (e.g., esophagus, colon, intestine, ileum, rectum, anus, liver, pancreas, stomach), genitourinary (e.g., bladder, kidney, prostate), musculoskeletal, nervous, pulmonary (e.g., lung), or reproductive (e.g., cervix, ovary, testicle) organ systems.

Interferons (IFN) are naturally occurring, hormone like proteins with anti-viral, antiproliferative, and immune-enhancing properties. They are produced by the body at low levels in response to viral infection or to other inducers. At least seven different protein subtypes of alpha interferon are present in ALFERON LDO®: IFN-α2, IFN-α4, IFN-α7, IFN-α8, IFN-α10, IFN-α16, and IFN-α17. ALFERON LDO® (low dose oral interferon alfa-n3 (human leukocyte derived)) is a different formulation of ALFERON N® interferon alfa-n3 (an injectable solution containing 5×10⁶ IU per ml), the only natural interferon currently FDA approved and available in the marketplace. Unlike recombinant IFN-α products which contain a single subspecies of IFN-α, interferon alfa-n3 contains at least seven species of IFN-α. They bind to the same receptors as interferon alfa-2b. ALFERON-LDO® interferon alfa-n3 consists of interferon alpha proteins about 166 amino acids in length and ranging in molecular weight from 16 Kda to 27 Kda. They include at least IFN-α2, IFN-α4, IFN-α7, IFN-α8, IFN-α10, IFN-α16, and IFN-α17. The specific activity of interferon alfa-n3 is at least about 2×10⁸ IU/mg of protein.

The IFN-α cocktail may be manufactured from pooled units of human blood. The manufacturing process includes isolation of buffy coat leukocytes, induction by incomplete infection with Sendai virus, immunoaffinity chromatography with a murine monoclonal antibody against multiple species of IFN-α, acidification (pH 2) for five days at 4° C. to inactivate and/or clear possible contaminating infectious agents (e.g., Sendai virus used for induction or blood borne viruses such as HIV-1, HTLV-I, HBV, HSV-1, hCMV, and EBV), and gel filtration chromatography. Alternatively, at least some IFN-α in the cocktail may be isolated from human blood product (e.g., whole blood or serum) or cultured medium in which mammalian cells produce human IFN-α protein of naturally-derived sequence and glycosylation pattern (e.g., conditioned medium). The purified cocktail may be used to formulate a medicament.

Formulations for oral administration include aqueous solutions, syrups, elixirs, powders, granules, tablets, and capsules which may contain excipients such as binding agents, fillers, stabilizers, disintegrants, wetting agents, suspending agents, emulsifying agents, preservatives, buffer salts, flavoring agents, coloring agents, sweetening agents, or any combination thereof. It may be a liquid, nebulized solution, aerosol spray, syrup, tablet, capsule, or lozenge. An orally administered formulation may contain sodium lactate as buffer, glycerol and/or xylitol as stabilizers, sodium benzoate as preservative, human albumin as carrier, and water as vehicle.

The IFN-α cocktail may be administered by any route involving oral, nasal, and/or buccal mucosa. The cocktail is preferably not administered by an enteral, parenteral, or inhalation route. It will be appreciated that the preferred dosage may vary with condition and age of the subject, the nature of the infectious or neoplastic disease, and the chosen IFN-α species. It is preferred to use highly purified mixtures of different human interferons (e.g., at least 80% or 85% or 90% or 95% of proteins in the cocktail are IFN-α species, not including intentionally added carrier proteins). The activity of the IFN-α cocktail may be at least 10⁶ IU/mg, at least 10⁷ IU/mg, or at least 10⁸ IU/mg of protein.

The dosage of the IFN-α cocktail will depend on the clinical status of the subject and the experience of the physician or veterinarian in treating the infection or tumor burden. It may be administered as an aqueous solution in the range from 5 IU per pound body weight/day to 1000 IU per pound body weight/day (alternatively, 25 IU to 200 IU per pound body weight/day). When calculated on the basis of a 150 pound human, this dosage is from 750 IU per day to 1.5×10⁵ IU per day. Our experience indicates beneficial results are obtained at dosage levels of α-interferon in excess of 750 IU per day, that is greater than 5 IU per pound body weight/day.

### Examples

### Example 1

Study A: asymptomatic HIV infected subjects with CD4 levels >400 were treated with 500 IU or 1,000 IU of ALFERON® in an aqueous buffered solution prepared by diluting ALFERON® for injection administered orally daily for 10 days. RNA from peripheral blood leukocytes was isolated from blood collected before, during and post-therapy using Paxgene technology for RNA isolation. A cDNA microarray analysis was utilized to identify genes which were modulated as a result of the ALFERON® oral dosing. Study B: normal healthy volunteers being studied in a similar manner.

The results demonstrate an induction of α-interferon related gene activity and differential gene modulation in peripheral blood leukocytes following the oral (mucosal) administration of 500 IU or 1,000 IU of a multi-species natural leukocyte α-interferon derived from human.

ALFERON® used in the study was supplied as an aqueous solution packaged in sealed polypropylene lined foil pouches. Each pouch contained 1.0 ml of ALFERON® (500 IU or 1,000 IU) or placebo. Solutions were taken orally each day for 10 days. No food or water is to be taken 30 minutes prior to through 30 minutes after administration to avoid enzymatic destruction of the polypeptide mixture. Dosing and blood sampling are shown in Table 1.

TABLE 1
Study Day Number and Event

| | Day Number | | | | | | | | | | | | | | | | |
|---|---|---|---|---|---|---|---|---|---|---|---|---|---|---|---|---|---|
| | 0* | 1 | 2 | 3 | 4 | 5 | 6 | 7 | 8 | 9 | 10 | 11 | 12 | 13 | 14 | 15 | 16 |
| LDO | | X | X | X | X | X | X | X | X | X | X | | | | | | |
| Dosing | | | | | | | | | | | | | | | | | |
| Blood | ↑ B1 + | | ↑ T1 | | | ↑ T2 | | | | | | ↑ T3 | ↑ T4 | | | | ↑ |
| Samples | B2 | | | | | | | | | | | | | | | | T5 |
| Drawn | | | | | | | | | | | | | | | | | |

*Day 0 = Baseline period in which two separate samples (B1 and B2) are drawn
Goal: Compare gene expression of T1-T5 Samples to two Baseline Samples Combined (i.e., B1 + B2)

Blood samples were subjected to cDNA microarray gene analysis as follows.

Array construction. The microarray used in this study comprised a subset of sequence verified cDNA' clones from the Research Genetics 40,000 clone set representing 950 genes containing adenylate/uridylate rich elements and 18 genes potentially involved in AU-directed mRNA decay, 855 ISGs representing an expansion of a previously described clone set containing confirmed and candidate genes stimulated by IFNs in diverse cell types, 288 genes responsive to poly(I)•poly(C), and 85 housekeeping genes.

Target RNA preparation. Target RNA was generated in a T7 polymerase based linear amplification reaction. Two μg total RNA and 5 pmol of T7-(dT)₂₄ primer 5′-GGCCAGTGAATTGTAATACGACTCACTATAGGGAGGCGG-(dT)₂₄-3′ (SEQ ID NO:1) in a total volume of 5.5 μl was incubated at 70° C. for 10 min and chilled on ice. For first strand cDNA synthesis, the annealed RNA template was incubated for 1 hr at 42° C. in a 10 μl reaction containing first strand buffer (Invitrogen), 10 mM DTT, 1 U per μl anti-RNase (Ambion), 500 μM dNTPs, and 2 U per μl Superscript II (Invitrogen). Second strand synthesis was for 2 hr at 16° C. in a total reaction volume of 50 μl containing first strand reaction products, second strand buffer (Invitrogen), 250 μM dNTPs, 0.06 U per μl DNA ligase (Ambion), 0.26 U per μl DNA polymerase I (New England Biolabs) and 0.012 U per μl RNase H (Ambion), followed by the addition of 3.3 U of T4 DNA polymerase (3 U per μl; New England Biolabs) and a further 15 min incubation at 16° C. Second strand reaction products were purified by phenol:chloroform:isoamyl alcohol extraction in Phaselock microcentrifuge tubes (Eppendorf) according to manufacturer's instructions and ethanol precipitated. In vitro transcription was performed using the T7 Megascript kit (Ambion) according to a modified protocol in which purified cDNA was combined with 1 μl each 10×ATP, GTP, CTP and UTP and 1 μl of T7 enzyme mix in a 10 μl reaction volume and incubated for 9 hr at 37° C. Amplified RNA was purified using the RNeasy purification kit (Ambion).

RNA labeling. Cy3 or Cy5 labeled cDNA was prepared by indirect incurporation. Two μg of amplified RNA, 1 μl dT₁₂₋₁₈ primer (1 μg per μl, Invitrogen), 2.6 μl random hexanucleotides (3 μg per μl, Invitrogen) and 1 μl anti-RNAse (Ambion) were combined in a reaction volume of 15.5 μl and incubated for 10 min at 70° C. Reverse transcription was for 2 hr at 42° C. in a 30 μl reaction containing annealed RNA template, first strand buffer, 500 mM each dATP, dCTP, dGTP, 300 μM dTTP, 200 μM aminoallyl-dUTP (Sigma), 10 mM DTT, and 12.7 U per μl Superscript II. For template hydrolysis, 10 μl of 0.1 M NaOH was added to the reverse transcription reaction and the mixture was incubated for 10 min at 70° C., allowed to cool at room temperature for 5 min and neutralized by addition of 10 μl of 0.1 M HCl. cDNA was precipitated at −20° C. for 30 min after addition of 1 μl of linear acrylamide (Ambion), 4 μl of 3 M NaOAc (pH 5.2) and 100 μl absolute ethanol then resuspended in 5 μl of 0.1 M NaHCO₃. For dye coupling, the contents of one tube of NHS ester containing Cy3 or Cy5 dye (Amersham Bio-sciences) was dissolved in 45 μl of DMSO. Five μl of dye solution was mixed with the cDNA and incubated for 1 hr in darkness at room temperature. Labeled cDNA was purified on a Qiaquick PCR purification column (Qiagen) according to manufacturer's instructions. Eluted cDNA was dried under vacuum and resuspended in 30 μl of Slidehyb II hybridization buffer (Ambion). After 2 min of denaturation at 95° C., the hybridization mixture was applied to the microarray slide under a coverslip. Hybridization proceeded overnight in a sealed moist chamber in a 55° C. water bath. Post-hybridization, slides were washed successively for 5 min each in 2×SSC/0.1% SDS at 55° C., then 2×SSC at 55° C. plus a final 5 min wash in 0.2×SSC at room temperature.

Data acquisition and normalization. Data were acquired with a GenePix 4000B laser scanner and GenePix Pro 5.0 software. Raw data were imported into GeneSpring 6.0 software (Silicon Genetics) and normalized based on the distribution of all values with locally weighted linear regression (LOESS) before further analysis.

The results demonstrate that orally administered Alferon® was well tolerated at the 500 and 1,000 IU/day dosage levels. cDNA microarray analysis identified 385 genes that were expressed greater than two fold over baseline in two or more patient samples (greater than 2-fold change over baseline is statistically reliable evidence of gene modulation by the test biological drug). An approximately four-fold increase in gene expression was seen at the 1,000 IU/day dosage level compared to 500 IU/day (p<0.0001). Although, not an exhaustive list, Table 2 shows 25 genes that were expressed greater than 2-fold over baseline in greater than or equal to 33% of patient samples. PDZ and LIN domain 5 and 2′-5′ oligo-adenylate synthetase-like were among the top five upregulated genes. 2′-5′ Oligo-adenylate synthetase is an important component of the interferon intracellular antiviral pathway. Importantly, genes related to activation of an inflammatory response such as tumor necrosis factor (TNF) related genes were actually down regulated, thus abrogating the harmful “cytokine storm” in disease processes.

TABLE 2
Genes Expressed ≧ Two Fold Over Baseline in ≧33% of Patient
Samples

| | | Expression Frequency (%) | | |
|---|---|---|---|---|
| Identified Gene | | 500 IU | 1,000 IU | Overall |
| 1 | SFRS protein kinase 1 | 83 | 40 | 59 |
| 2 | Homo sapiens, clone image: 5164031, mRNA | 17 | 87 | 56 |
| 3 | PDZ and LIN domain 5 | 0 | 93 | 52 |
| 4 | Interleukin 17 receptor | 0 | 93 | 52 |
| 5 | 2′-5′ oligoadenylate synthetase-like | 33 | 67 | 52 |
| 6 | Similar to KIAA0160 gene product | 0 | 87 | 48 |
| 7 | N-myristoyltransferase 2 | 0 | 80 | 44 |
| 8 | Proteasome (prosome, macropain) 265 subunit, | 0 | 73 | 41 |
| | ATPase, 6 | | | |
| 9 | Coagulation factor II (thrombin) receptor | 0 | 73 | 41 |
| 10 | Cytochrome P450, family 51, subfamily A, | 0 | 73 | 41 |
| | polypeptide | | | |
| 11 | Interferon induced transmembrane protein 2 | 33 | 47 | 41 |
| 12 | Major histocompatibility complex, class I, F | 33 | 47 | 41 |
| 13 | Sarcoglycan, beta (43 kDa dystrophin-associated | 0 | 73 | 41 |
| | glycoprotein) | | | |
| 14 | Glutamate dehydrogenase 1 | 0 | 73 | 41 |
| 15 | FGG | 0 | 67 | 37 |
| 16 | Coagulation factor III (thromboplastin, tissue factor) | 0 | 67 | 37 |
| 17 | Interferon (alpha, beta, and omega) receptor 1 | 0 | 67 | 37 |
| 18 | Ribosomal protein S6 kinase, 90 KDa, polypeptide 3 | 0 | 60 | 33 |
| 19 | Hemoglobin, epsilon 1 | 33 | 33 | 33 |
| 20 | Acyl-coenzyme A dehydrogenase, short/branched | 0 | 60 | 33 |
| | chain | | | |
| 21 | Hypothetical protein MGC20481 | 17 | 47 | 33 |
| 22 | RAB7, member RAS oncogene family | 17 | 47 | 33 |
| 23 | Ribosomal protein S15a | 0 | 60 | 33 |
| 24 | Glutamate dehydrogenase I | 0 | 60 | 33 |
| 25 | Small nuclear RNA activating complex, polypeptide 3, | 25 | 40 | 33 |
| | 50 KDa | | | |

### Example 2

Cynomolgus macaques (Macaca fascicularis) were infected with influenza virus H5N1, demonstrated that clinical signs in the macaques resemble those found in humans infected with the avian influenza H5N1 viruses, thus allowing the infection of cynomolgus macaques with influenza H5N1 viruses to serve as a model for these infections in humans (Rimmelzwaan et al. Avian Dis. 47(3 Suppl):931-933, 2003; Kuiken et al. Vet. Pathol. 40:304-310, 2003; Rimmelzwaan et al. J. Virol. 75:6687-6691, 2001).

The macaque H5N1 infection model described above was used to determine the prophylactic efficacy of Alferon® in cynomolgus macaques following oro-mucosal delivery of Alferon® starting five days before intratracheal challenge with influenza virus A/Vietnam/1194/'04 (H5N1), and followed by daily dosing for ten days at various doses.

Animals were anesthetized before each procedure for safety and practical reasons (oro-mucosal delivery of Alferon® and infection with influenza virus H5N1). The experiment consisted of four groups of three animals each. Group A was treated with 10 mg/kg Alferon®, group B with 25 mg/kg Alferon®, group C with 62.5 mg/kg Alferon®, and group D with placebo. There were no adverse effects observed.

Upon euthanasia at day 5 after infection, macroscopic lung lesions indicated that animals from group C treated with 62.5 mg/kg Alferon® showed no separated dark red area(s) or diffuse dark areas on the lungs in contrast to animals of the other groups. This is consistent with the microscopic findings which indicate also a lower grade of primary atypical pneumonia in both left cranial and caudal lung lobes in animals of this group.

Prophylactic treatment of macaques with oro-mucosal delivery of Alferon® appears to have a beneficial dose dependent effect with reduced gross and histo pathology in treated animals.

Study A: asymptomatic HIV infected subjects with CD4 levels greater than 400 were treated with 500 IU or 1,000 IU of Alferon® in an aqueous buffered solution prepared by diluting Alferon® for injection administered orally daily for 10 days. RNA from peripheral blood leukocytes was isolated from blood collected before, during and post-therapy using Paxgene technology for RNA isolation. A cDNA microarray analysis was utilized to identify genes which were modulated as a result of the Alferon® oral dosing. Study B: normal healthy volunteers being studied in a similar manner.

Initial results in Study A demonstrate induction of α-interferon related gene activity in peripheral blood leukocytes following the oral administration of 500 IU or 1,000 IU of a multi-species natural leukocyte α-interferon.

Alferon® used in the study was supplied as a liquid solution packaged in sealed polypropylene lined foil pouches. Each pouch contained 1.0 ml of Alferon® (500 IU or 1,000 IU) or placebo. Solutions were taken orally each day for 10 days. No food or water is taken 30 minutes prior to through 30 minutes after administration. Dosing and blood sampling are shown in Table 3. Dose effects are shown in Tables 4 to 6.

TABLE 3
Study Day Number and Event

| | Day Number | | | | | | | | | | | | | | | | |
|---|---|---|---|---|---|---|---|---|---|---|---|---|---|---|---|---|---|
| | 0* | 1 | 2 | 3 | 4 | 5 | 6 | 7 | 8 | 9 | 10 | 11 | 12 | 13 | 14 | 15 | 16 |
| LDO | | X | X | X | X | X | X | X | X | X | X | | | | | | |
| Dosing | | | | | | | | | | | | | | | | | |
| Blood | ↑ B1 + | | ↑ T1 | | | ↑ T2 | | | | | | ↑ T3 | ↑ T4 | | | | ↑ T5 |
| Samples | B2 | | | | | | | | | | | | | | | | |
| Drawn | | | | | | | | | | | | | | | | | |

*Day 0 = Baseline period in which two separate samples (B1 and B2) are drawn
Goal: Compare gene expression of T1-T5 Samples to two Baseline Samples Combined (i.e., B1 + B2)

Blood samples were subjected to cDNA microarray gene analysis as follows.

Array construction. The microarray used in this study comprised a subset of sequence verified cDNA clones from the Research Genetics 40,000 clone set representing 950 genes containing adenylate/uridylate rich elements and 18 genes potentially involved in AU-directed mRNA decay, 855 ISGs representing an expansion of a previously described clone set containing confirmed and candidate genes stimulated by IFNs in diverse cell types, 288 genes responsive to poly(I) poly(C), and 85 housekeeping genes.

Target RNA preparation. Target RNA was generated in a T7 polymerase based linear amplification reaction. Two μg total RNA and 5 pmol of T7-(dT)₂₄ primer 5′-GGCCAGTGAATTGTAATACGACTCACTATAGGGAGGCGG-(dT)₂₄-3′ (SEQ ID NO:1) in a total volume of 5.5 μl was incubated at 70° C. for 10 min and chilled on ice. For first strand cDNA synthesis, the annealed RNA template was incubated for 1 hr at 42° C. in a 10 μl reaction containing first strand buffer (Invitrogen), 10 mM DTT, 1 U per μl anti-RNase (Ambion), 500 μM dNTPs, and 2 U per μl Superscript II (Invitrogen). Second strand synthesis was for 2 hr at 16° C. in a total reaction volume of 50 μl containing first strand reaction products, second strand buffer (Invitrogen), 250 μM dNTPs, 0.06 U per μl DNA ligase (Ambion), 0.26 U per μl DNA polymerase I (New England Biolabs) and 0.012 U per μl RNase H (Ambion), followed by the addition of 3.3 U of T4 DNA polymerase (3 U per μl; New England Biolabs) and a further 15 min incubation at 16° C. Second strand reaction products were purified by phenol:chloroform:isoamyl alcohol extraction in Phaselock microcentrifuge tubes (Eppendorf) according to manufacturer's instructions and ethanol precipitated. In vitro transcription was performed using the T7 Megascript kit (Ambion) according to a modified protocol in which purified cDNA was combined with 1 μl each 10×ATP, GTP, CTP and UTP and 1 μl of T7 enzyme mix in a 10 μl reaction volume and incubated for 9 hr at 37° C. Amplified RNA was purified using the Rneasy purification kit (Ambion).

RNA labeling. Cy3 or Cy5 labeled cDNA was prepared by indirect incorporation. Two μg of amplified RNA, 1 μl dT₁₂₋₁₈ primer (1 μg per μl, Invitrogen), 2.6 μl random hexanucleotides (3 μg per μl, Invitrogen) and 1 μl anti-RNAse (Ambion) were combined in a reaction volume of 15.5 μl and incubated for 10 min at 70° C. Reverse transcription was for 2 hr at 42° C. in a 30 μl reaction containing annealed RNA template, first strand buffer, 500 mM each dATP, dCTP, dGTP, 300 μM dTTP, 200 μM aminoallyl-dUTP (Sigma), 10 mM DTT, and 12.7 U per μl Superscript II. For template hydrolysis, 10 μl of 0.1 M NaOH was added to the reverse transcription reaction and the mixture was incubated for 10 min at 70° C., allowed to cool at room temperature for 5 min and neutralized by addition of 10 μl of 0.1 M HCl. cDNA was precipitated at −20° C. for 30 min after addition of 1 μl of linear acrylamide (Ambion), 4 μl of 3 M NaOAc (pH 5.2) and 100 μl absolute ethanol then resuspended in 5 μl of 0.1 M NaHCO₃. For dye-coupling, the contents of one tube of NHS ester containing Cy3 or Cy5 dye (Amersham Biosciences) was dissolved in 45 μl DMSO. Five μl of dye solution was mixed with the cDNA and incubated for 1 hr in darkness at room temperature. Labeled cDNA was purified on a Qiaquick PCR purification column (Qiagen) according to manufacturer's instructions. Eluted cDNA was dried under vacuum and resuspended in 30 μl of Slidehyb II hybridization buffer (Ambion). After 2 min of denaturation at 95° C., the hybridization mixture was applied to the microarray slide under a coverslip. Hybridization proceeded overnight in a sealed moist chamber in a 55° C. water bath. Post-hybridization, slides were washed successively for 5 min each in 2×SSC/0.1% SDS at 55° C., then 2×SSC at 55° C. plus a final 5 min wash in 0.2×SSC at room temperature.

Data acquisition and normalization. Data were acquired with a GenePix 4000B laser scanner and GenePix Pro 5.0 software. Raw data were imported into GeneSpring 6.0 software (Silicon Genetics) and normalized based on the distribution of all values with locally weighted linear regression (LOESS) before further analysis.

Initial results in Study A demonstrate that orally administered Alferon® was well tolerated at the 500 and 1,000 IU/day dosage levels. cDNA microarray analysis identified 385 genes that were expressed greater than two fold over baseline in two or more patient samples. As shown in Tables 4 to 6, an approximately four-fold increase in gene expression was seen at the 1,000 IU/day dosage level compared to 500 IU/day (p<0.0001). Although, not an exhaustive list, Table 7 shows 25 genes that were expressed greater than 2-fold over baseline in greater than or equal to 33% of patient samples. PDZ and LIN domain 5 and 2′-5′ oligo-adenylate synthetase-like were among the top five upregulated genes. 2′-5′ Oligo-adenylate synthetase is an important component of the interferon intracellular antiviral pathway. Importantly, as shown in Table 8, genes related to activation of an inflammatory response such as tumor necrosis factor (TNF) related genes were down regulated.

Recent evidence shows that the virulence of influenza A including avian (H5N1) isolates correlates with the ability of the non-structural NS1 viral protein to bind to human PDZ domains and thereby abrogating the expression of antiviral genes in host cells including interferon pathways. Thus, the finding that orally administered Alferon® can upregulate PDZ domain expression raises the possibility that Alferon® could have an important role in abrogating the ability of influenza viruses including avian (H5N1) to evade human host defense mechanisms.

Orally administered Alferon® was well-tolerated with no serious adverse events reported. Only several mild, adverse events were reported, such as a metallic taste in mouth or flatulence or bloating. There were no clinically significant changes in laboratory parameters and no changes in Karnofsky Performance Status (KPS).

Experiments to date indicate that a biological cocktail of natural human interferon species administered orally has systemic biological activity based on upregulation of α-interferon related genes in peripheral blood leukocytes. Because alpha α-interferon are broad spectrum antiviral or immunomodulatory molecules, potential applications in numerous α-interferon-sensitive diseases exist, including application to respiratory infections such as avian influenza.

TABLE 4
Dose Effect: Number of Genes with Expression Increased ≧2 Fold
Over Baseline in Two or More Patient Samples

| | Dose | | | | | | | | |
|---|---|---|---|---|---|---|---|---|---|
| | 500 IU | | | | 1,000 IU | | | | Fold |
| | Patient # | | | | | | | | Increase |
| | 1 | 2 | 3 | Mean | 4 | 5 | 6 | Mean | of Mean |
| Day 2 | 10 | 1 | 39 | 16.7 | 85 | 77 | 108 | 90 | 5.4 |
| Day 5 | 14 | 4 | 23 | 13.7 | 54 | 72 | 35 | 54 | 3.9 |
| Day 11 | 1 | 8 | 4 | 4.3 | 4 | 45 | 40 | 30 | 6.9 |
| Day 12 | 3 | 15 | — | 9.0 | 19 | 44 | 28 | 30 | 3.4 |
| Day 16 | — | 14 | — | 14 | 3 | 59 | 48 | 37 | 2.6 |
| Mean | 7.0 | 8.4 | 22 | 12.5 | 33 | 59 | 52 | 48 | 3.9 |

Student's t-test, p-value <0.0001 (n = 385)

TABLE 5
Dose Effect: Number of Genes with Expression Increased ≧2 Fold
Over Baseline in Three or More Patient Samples

| | Dose | | | | | | | | |
|---|---|---|---|---|---|---|---|---|---|
| | 500 IU | | | | 1,000 IU | | | | Fold |
| | Patient # | | | | | | | | Increase |
| | 1 | 2 | 3 | Mean | 4 | 5 | 6 | Mean | of Mean |
| Day 2 | 3 | 1 | 19 | 7.6 | 36 | 41 | 42 | 39.7 | 5.2 |
| Day 5 | 2 | 2 | 6 | 3.3 | 16 | 17 | 16 | 16.3 | 4.9 |
| Day 11 | 1 | 1 | 1 | 1.0 | 3 | 3 | 3 | 3.0 | 3.0 |
| Day 12 | 1 | 2 | — | 1.5 | 7 | 8 | 8 | 7.7 | 5.1 |
| Day 16 | — | 0 | — | 0 | 2 | 2 | 2 | 2.0 | >5 |
| Mean | 1.8 | 1.2 | 8.7 | 3.9 | 12.8 | 14.2 | 14.2 | 13.7 | 3.5 |

Student's t-test, p-value <0.0001 (n = 252)

TABLE 6
Dose Effect: Number of Genes with Expression Increased ≧3 Fold
Over Baseline in Two or More Patient Samples

| | Dose | | | | | | | | |
|---|---|---|---|---|---|---|---|---|---|
| | 500 IU | | | | 1,000 IU | | | | Fold |
| | Patient # | | | | | | | | Increase |
| | 1 | 2 | 3 | Mean | 4 | 5 | 6 | Mean | of Mean |
| Day 2 | 0 | 0 | 9 | 3.0 | 23 | 10 | 37 | 23.0 | 7.8 |
| Day 5 | 5 | 1 | 5 | 3.7 | 16 | 19 | 4 | 13.0 | 3.5 |
| Day 11 | 1 | 2 | 0 | 1.0 | 0 | 14 | 3 | 5.7 | 5.7 |
| Day 12 | 1 | 3 | — | 2.0 | 3 | 10 | 2 | 5.0 | 2.5 |
| Day 16 | — | 0 | — | 0.0 | 0 | 14 | 6 | 6.7 | >5 |
| Mean | 1.8 | 1.2 | 4.7 | 2.6 | 8.4 | 13.4 | 10.4 | 10.7 | 4.1 |

Student's t-test, p-value <0.0001 (n = 69)

TABLE 7
Genes Expressed ≧ Two Fold Over Baseline in ≧ 33% of Patient
Samples

| | | Expression Frequency (%) | | |
|---|---|---|---|---|
| Identified Gene | | 500 IU | 1,000 IU | Overall |
| 1 | SFRS protein kinase 1 | 83 | 40 | 59 |
| 2 | Homo sapiens, clone image: 5164031, mRNA | 17 | 87 | 56 |
| 3 | PDZ and LIN domain 5 | 0 | 93 | 52 |
| 4 | Interleukin 17 receptor | 0 | 93 | 52 |
| 5 | 2′-5′ oligoadenylate synthetase-like | 33 | 67 | 52 |
| 6 | Similar to KIAA0160 gene product | 0 | 87 | 48 |
| 7 | N-myristoyltransferase 2 | 0 | 80 | 44 |
| 8 | Proteasome (prosome, macropain) 265 subunit, | 0 | 73 | 41 |
| | ATPase, 6 | | | |
| 9 | Coagulation factor II (thrombin) receptor | 0 | 73 | 41 |
| 10 | Cytochrome P450, family 51, subfamily A, | 0 | 73 | 41 |
| | polypeptide | | | |
| 11 | Interferon induced transmembrane protein 2 | 33 | 47 | 41 |
| 12 | Major histocompatibility complex, class I, F | 33 | 47 | 41 |
| 13 | Sarcoglycan, beta (43 kDa dystrophin-associated | 0 | 73 | 41 |
| | glycoprotein) | | | |
| 14 | Glutamate dehydrogenase 1 | 0 | 73 | 41 |
| 15 | FGG | 0 | 67 | 37 |
| 16 | Coagulation factor III (thromboplastin, tissue factor) | 0 | 67 | 37 |
| 17 | Interferon (alpha, beta, and omega) receptor 1 | 0 | 67 | 37 |
| 18 | Ribosomal protein S6 kinase, 90 KDa, polypeptide 3 | 0 | 60 | 33 |
| 19 | Hemoglobin, epsilon 1 | 33 | 33 | 33 |
| 20 | Acyl-coenzyme A dehydrogenase, short/branched | 0 | 60 | 33 |
| | chain | | | |
| 21 | Hypothetical protein MGC20481 | 17 | 47 | 33 |
| 22 | RAB7, member RAS oncogene family | 17 | 47 | 33 |
| 23 | Ribosomal protein S15a | 0 | 60 | 33 |
| 24 | Glutamate dehydrogenase I | 0 | 60 | 33 |
| 25 | Small nuclear RNA activating complex, polypeptide | 25 | 40 | 33 |
| | 3, 50 KDa | | | |

TABLE 8
Tumor Necrosis Factor (TNF) Related Genes with ≧ 50% Reduction in
Expression

TNF (ligand) superfamily, member 11
TNF receptor superfamily, member 6b, decoy
TNF receptor - associated factor 1
TNF, alpha-induced protein 6
TNF receptor superfamily, member 10b

Patents, patent applications, books, and other publications cited herein are incorporated by reference in their entirety.

In stating a numerical range, it should be understood that all values within the range are also described (e.g., one to ten also includes every integer value between one and ten as well as all intermediate ranges such as two to ten, one to five, and three to eight). The term “about” may refer to the statistical uncertainty associated with a measurement or the variability in a numerical quantity which a person skilled in the art would understand does not affect operation of the invention or its patentability.

All modifications and substitutions that come within the meaning of the claims and the range of their legal equivalents are to be embraced within their scope. A claim which recites “comprising” allows inclusion of other elements to be within the scope of the claim; the invention is also described by such claims reciting the transitional phrases “consisting essentially of” (i.e., allowing the inclusion of other elements to be within the scope of the claim if they do not materially affect operation of the invention) or “consisting of” (i.e., allowing only the elements listed in the claim other than impurities or inconsequential activities which are ordinarily associated with the invention) instead of the “comprising” term. Any of these three transitions can be used to claim the invention.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. A method of treating a subject infected with a bacterium or protozoan, said method comprising administration to the subject of a pharmaceutical composition comprised of a purified mixture of at least three different human interferon-alpha proteins with native amino acid sequences and glycosylation patterns in an amount sufficient to reduce or eliminate infection of the subject by the bacterium or protozoan, wherein the pharmaceutical composition is applied oropharyngeally and in a dosage from 5 IU per pound body weight/day to 1000 IU per pound body weight/day.

2. The method according to claim 1, wherein the subject is infected by a bacterium.

3. The method according to claim 1, wherein the subject is infected by a protozoan.

4. A method of treating a subject bearing a tumor or other transformed cell, said method comprising administration to the subject of a pharmaceutical composition comprised of a purified mixture of at least three different human interferon-alpha proteins with native amino acid sequences and glycosylation patterns in an amount sufficient to reduce or eliminate proliferation of the tumor or other transformed cell in the subject, wherein the pharmaceutical composition is applied oropharyngeally and in a dosage from 5 IU/pound body weight per day to 1000 IU/pound body weight per day.

5. The method according to claim 4, wherein the subject has a cancer initiated by virus infection.

6. A method of treating a subject infected by a bacterium or protozoa or affected by a pathogenic disease process susceptible to treatment by specific genes regulated by interferon-alpha protein, said method comprising administration to the subject of a pharmaceutical composition comprised of a purified mixture of at least three different human interferon-alpha proteins with native amino acid sequences and glycosylation patterns in an amount sufficient to increase expression of genes which are negatively regulated by the infection or disease process and to decrease expression of genes which are positively regulated by the infection or disease process, wherein the pharmaceutical composition is applied oropharyngeally and in a dosage from 5 IU/pound body weight per day to 1000 IU/pound body weight per day.

7. The method according to claim 1, wherein the subject is a human.

8. The method according to claim 1, wherein the pharmaceutical composition is ALFERON® LDO interferon alfa-n3.

9. The method according to claim 7, wherein the pharmaceutical composition is ALFERON® LDO interferon alfa-n3.

10. The method according to claim 1, wherein at least cytokine production or co-stimulatory molecule signaling which had been initiated by the microbe or processes resulting in autoimmunity or neurodegeneration in the subject is remodulated by the mixture of different human interferon-alpha proteins.

11. Use of a purified mixture of at least three different human interferon-alpha proteins with native amino acid sequences and glycosylation patterns to manufacture a medicament applied oropharyngeally and in a dosage from 5 IU per pound body weight/day to 1000 IU per pound body weight/day for treating bacterial infection, protozoal infection, or cancer.
